(19) United States
(12) Patent Application Publication
(10) Pub. No.: US 2025/0072842 A1
(43) Pub. Date: Mar. 6, 2025

(54) ELECTRONIC DEVICE FOR DISPLAYING INFORMATION RELATED TO WEARABLE ELECTRONIC DEVICE, OPERATION METHOD THEREOF, AND RECORDING MEDIUM

(71) Applicant: Samsung Electronics Co., Ltd., Suwon-si (KR)

(72) Inventors: Hyoujoo KWON, Suwon-si (KR); Hyuncheol PARK, Suwon-si (KR); Soohan YOO, Suwon-si (KR); Seungwon LEE, Suwon-si (KR); Hyunjun JUNG, Suwon-si (KR); Seongwook JO, Suwon-si (KR)

(21) Appl. No.: 18/819,855

(22) Filed: Aug. 29, 2024

Related U.S. Application Data
(63) Continuation of application No. PCT/KR2024/012708, filed on Aug. 26, 2024.

(30) Foreign Application Priority Data
Aug. 29, 2023 (KR) 10-2023-0113386
Sep. 20, 2023 (KR) 10-2023-0125816

(51) Int. Cl. A61B 5/00; G06F 3/16; H02J 7/00

(52) U.S. Cl. CPC A61B 5/7445; A61B 5/6826; A61B 5/749; G06F 3/167; H02J 7/0047

(57) ABSTRACT

According to an embodiment, an electronic device may include a first battery, charging circuitry, communication circuitry, a display, and at least one processor, wherein the at least one processor is configured to, based on identifying that a wearable electronic device being mounted in the electronic device, wirelessly transmit power to the wearable electronic device using the charging circuitry; display, on the display, at least one of first state information of the first battery and second state information of a second battery included in the wearable electronic device while wirelessly transmitting the power; display at least one object corresponding to at least one function related to the wearable electronic device on the display; and based on identifying an input to a first object among the at least one object, transmit a control signal for causing the wearable electronic device to execute a first function corresponding to the first object to the wearable electronic device through the communication circuitry.

### CROSS-REFERENCE TO RELATED APPLICATIONS

This application is a continuation of International Application No. PCT/KR2024/012708 designating the United States, filed on Aug. 26, 2024, in the Korean Intellectual Property Receiving Office and claiming priority to Korean Patent Application Nos. 10-2023-0113386, filed on Aug. 29, 2023, and 10-2023-0125816, filed on Sep. 20, 2023, in the Korean Intellectual Property Office, the disclosures of each of which are incorporated by reference herein in their entireties.

### BACKGROUND

### Field

The disclosure relates to an electronic device for displaying information related to a wearable electronic device, an operation method thereof, and a recording medium.

### Description of Related Art

Recently, the use of an electronic device, such as a smartphone, a tablet personal computer (PC), or a wearable device, that can be easily carried has increased, and as the use of such electronic devices has been sharply increased, the electronic devices are being developed to be wearable on a user in order to enhance portability and user accessibility. An example of such an electronic device includes a wearable electronic device.

The wearable electronic device may be implemented as a smart watch which can be worn on the wrist of the user, a smart ring which can be worn on a finger of the user, and a head mounted device which can be worn on the head of the user.

The above-described information may be provided as related art for the purpose of helping understanding of the disclosure. No claim or determination is made as to whether any of the foregoing may be applied as prior art regarding the disclosure.

### SUMMARY

According to an embodiment, an electronic device may include a first battery, charging circuitry, communication circuitry, a display, at least one processor, and memory storing instructions.

According to an embodiment, the electronic device may transmit, based on identifying that a wearable electronic device is mounted in the electronic device, using the charging circuitry, power wirelessly to the wearable electronic device.

According to an embodiment, the electronic device may display, on the display, at least one of first state information of the first battery or second state information of a second battery included in the wearable electronic device while transmitting the power wirelessly.

According to an embodiment, the electronic device may display, on the display, at least one object corresponding to at least one function related to the wearable electronic device.

According to an embodiment, the electronic device may transmit, based on identifying an input to a first object among the at least one object, through the communication circuitry, a control signal to cause the wearable electronic device to execute a first function corresponding to the first object to the wearable electronic device.

According to an embodiment, a method of operating an electronic device may include based on identifying that a wearable electronic device is mounted in the electronic device, wirelessly transmitting, using charging circuitry included in the electronic device, power to the wearable electronic device.

According to an embodiment, the method of operating an electronic device may include displaying, on a display, at least one of first state information of a first battery included in the electronic device or second state information of a second battery included in the wearable electronic device while wirelessly transmitting the power.

According to an embodiment, the method of operating an electronic device may include displaying, on the display, at least one object corresponding to at least one function related to the wearable electronic device.

According to an embodiment, the method of operating an electronic device may include based on identifying an input to a first object among the at least one object, transmitting a control signal for causing the wearable electronic device to execute a first function corresponding to the first object to the wearable electronic device through communication circuitry included in the electronic device.

According to an embodiment, a non-transitory computer-readable recording medium may store instructions which, when executed by at least one processor of an electronic device, individually and/or collectively, cause the electronic device to perform at least one operation including, based on identifying that a wearable electronic device is mounted in an electronic device, wirelessly transmitting, using charging circuitry included in the electronic device, power to the wearable electronic device.

According to an embodiment, the non-transitory computer-readable recording medium may store instructions which, when executed by at least one processor of an electronic device, individually and/or collectively, cause the electronic device to perform at least one operation including, displaying, on a display, at least one of first state information of a first battery included in the electronic device or second state information of a second battery included in the wearable electronic device while wirelessly transmitting the power.

According to an embodiment, the non-transitory computer-readable recording medium may store instructions which, when executed by at least one processor of an electronic device, individually and/or collectively, cause the electronic device to perform at least one operation including, displaying, on the display included in the electronic device, at least one object capable of executing at least one function related to the wearable electronic device.

According to an example embodiment, the non-transitory computer-readable recording medium may store instructions which, when executed by at least one processor of an electronic device, individually and/or collectively, cause the electronic device to perform at least one operation including, based on identifying an input to a first object among the at least one object, transmitting a control signal for causing the wearable electronic device to execute a first function corresponding to the first object to the wearable electronic device through communication circuitry included in the electronic device.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an electronic device in a network environment according to various embodiments.

FIG. 2 illustrates a state in which a wearable electronic device is mounted in an electronic device according to an embodiment.

FIG. 3 is a block diagram illustrating a system including an electronic device and a wearable electronic device according to an embodiment.

FIG. 4 is a flow chart illustrating an operation of displaying state information of a battery by an electronic device according to an embodiment.

FIG. 5 is a flow chart illustrating an operation of transmitting a control signal to a wearable electronic device by an electronic device to execute a function of the wearable electronic device according to an embodiment.

FIG. 6A is a flow chart illustrating an operation of displaying biometric information by an electronic device when a wearable electronic device is mounted in the electronic device according to an embodiment.

FIG. 6B is a flow chart illustrating an operation of displaying biometric information by an electronic device when a wearable electronic device is not mounted in the electronic device according to an embodiment

FIG. 7 is a flow chart illustrating an operation of transmitting, to a wearable electronic device by an electronic device, a control signal for causing information on a notification for charging a second battery of the wearable electronic device to be output according to an embodiment.

FIG. 8 is a flow chart illustrating an operation of identifying, by an electronic device, whether a user wearing a wearable electronic device is working out according to an embodiment.

FIG. 9A is a flow chart illustrating an operation of transmitting, by an electronic device, a control signal for causing a measurement period of a sensor of a wearable electronic device to be adjusted according to an embodiment.

FIG. 9B is a flow chart illustrating an operation of displaying biometric information of a user by an electronic device when a wearable electronic device is configured to be in a sleep mode according to an embodiment.

FIG. 10 illustrates a screen of displaying at least one object corresponding to at least one function related to a wearable electronic device on a display by an electronic device according to an embodiment.

FIG. 11A illustrates a screen for displaying state information of a second battery of a wearable electronic device on a display by an electronic device according to an embodiment.

FIG. 11B illustrates a screen for displaying first state information of a first battery and second state information of a second battery of an electronic device on a display by the electronic device according to an embodiment.

FIG. 12A illustrates a screen for displaying a heart rate of a user wearing a wearable electronic device on a display by an electronic device according to an embodiment.

FIG. 12B illustrates a screen for displaying a heart rate of a user on a display by an electronic device in a state in which a wearable electronic device is mounted in the electronic device according to an embodiment.

FIG. 13A illustrates a screen for displaying the number of steps of a user wearing a wearable electronic device on a display by an electronic device according to an embodiment.

FIG. 13B illustrates a screen for displaying the number of steps of a user on a display by an electronic device in a state in which a wearable electronic device is mounted in the electronic device according to an embodiment.

FIG. 14 illustrates a screen for displaying a skin temperature and a fertile window of a user wearing a wearable electronic device on a display by an electronic device according to an embodiment.

FIG. 15 illustrates a screen for displaying sleeping hours of a user wearing a wearable electronic device on a display by an electronic device according to an embodiment.

FIG. 16 illustrates a screen for displaying biometric information of a user wearing a wearable electronic device on a display by an electronic device according to an embodiment.

FIG. 17 illustrates a state in which an external electronic device is worn on a wrist part of a user and a wearable electronic device is worn on a finger part of the user according to an embodiment.

### DETAILED DESCRIPTION

Hereinafter, embodiments of the disclosure are described with reference to the accompanying drawings to allow those skilled in the art to which the disclosure belongs to easily implement the same. However, the disclosure may be implemented in various different forms, and is not limited to the embodiment described herein. In relation to the description of drawings, the same or similar reference numerals may be used for the same or similar elements. In addition, in the drawings and the description related thereto, description of a well-known function and configuration may be omitted for the purpose of clarity and brevity.

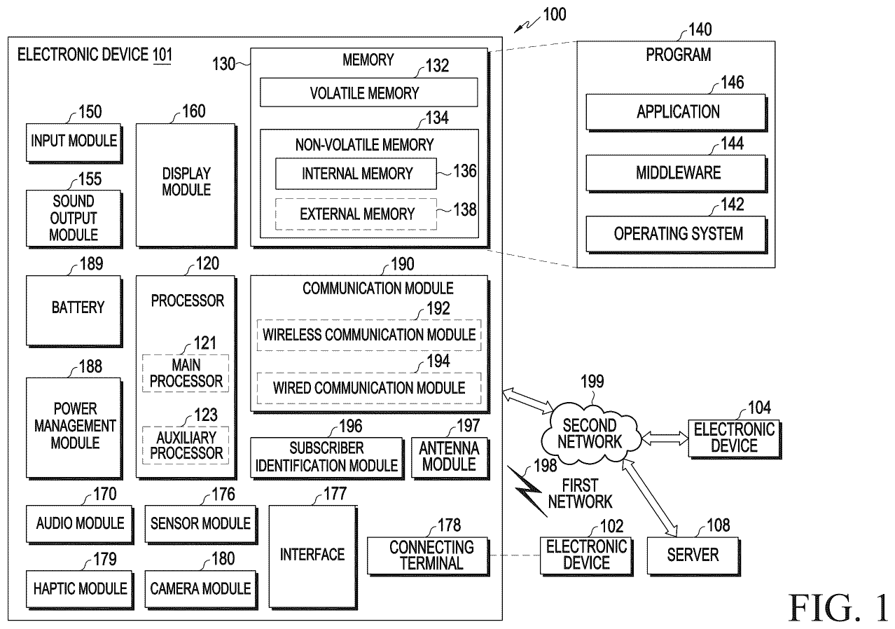

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions. The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to an embodiment, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

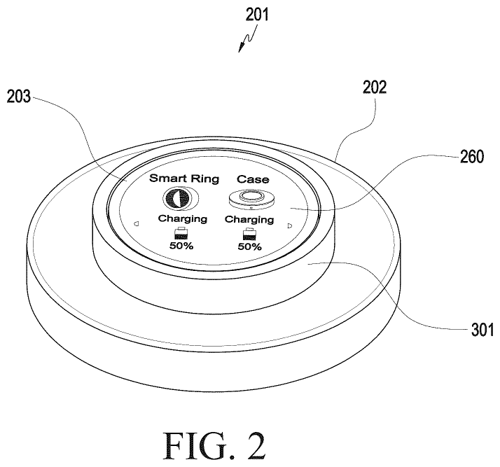

FIG. 2 illustrates a state in which a wearable electronic device is mounted in an electronic device according to an embodiment.

Referring to FIG. 2, according to an embodiment, an electronic device 201 may be implemented as a cradle or a case. For example, the electronic device 201 may be implemented in the form enabling a wearable electronic device 301 to be mounted or stored (e.g., a cradle shape or a case shape) therein. The wearable electronic device 301 may be implemented as a wearable electronic device (e.g., a wearable electronic device having a ring shape) to be mountable in the electronic device 201.

According to an embodiment, the wearable electronic device 301 may be worn on a finger part of a user. However, this is simply an example, and the electronic device 201 and the wearable electronic device 301 may be implemented as various types of electronic devices.

According to an embodiment, the electronic device 201 may include a first housing 202 and a second housing 203 disposed on the first housing 202. According to an embodiment, the area of the first housing 202 may be greater than that of the second housing 203.

According to an embodiment, the wearable electronic device 301 may be mounted on the external surface of the first housing 203.

According to an embodiment, the electronic device 201 may wirelessly charge the wearable electronic device 301 when the wearable electronic device 301 is identified to be mounted on the external surface of the second housing 203.

According to an embodiment, the wearable electronic device 301 may be implemented as a ring-shaped wearable electronic device which can be worn on a finger of the user. The wearable electronic device 301 may not include an output means such as a display and/or a speaker due to spatial constraints. The wearable electronic device 301 may not be able to output information (e.g., battery information) on the wearable electronic device by itself. In addition, even though the wearable electronic device 301 obtains or detects biometric information or motion information of the user, the wearable electronic device 301 may not be able to output the corresponding information by itself.

According to an embodiment, the wearable electronic device 301 may not include an input means such as a physical button, a soft button, a touchscreen, and/or a microphone due to spatial constraints. The wearable electronic device 301 may include only a limited number of input means (e.g., a physical button and/or soft button) due to spatial constraints. However, due to the limited input means, the user may experience inconvenience in executing or controlling a function related to the wearable electronic device 301 through the wearable electronic device 301.

In the conventional art, an electronic device (e.g., an electronic device having a case shape) was able to execute only a function of charging a wearable electronic device mounted therein. For example, the conventional electronic device did not include an output such as a display (or a touchscreen) and/or a speaker. Accordingly, the conventional electronic device was not able to visually or acoustically provide information related to the wearable electronic device and information related to the electronic device to the user. In addition, the conventional electronic device did not include an input means such as a physical button, a soft button, a touchscreen, and/or a microphone. Accordingly, the conventional electronic device was not able to execute or control a function related to the wearable electronic device. The conventional electronic device was able to execute or control only limited functions among the functions related to the wearable electronic device.

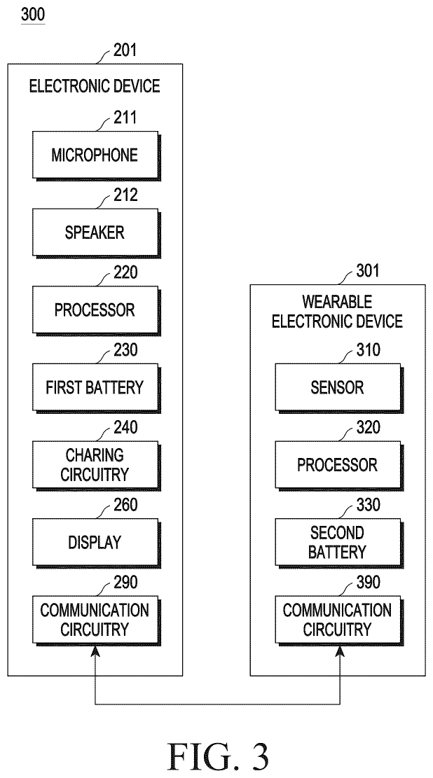

To solve the problems above, according to an embodiment, the electronic device 201 may include an input means and/or an output means such as a display (e.g., a display 260 of FIG. 3), a microphone (e.g., a microphone 211 of FIG. 3), and a speaker (e.g., a speaker 212 of FIG. 3). According to the implementation, the electronic device 201 may further include a physical button and/or a soft button. The electronic device 201 may output state information of the wearable electronic device 301, or output the user's biometric information and/or motion information obtained by the wearable electronic deice 301. In addition, the electronic device 201 may execute and/or control the function of the wearable electronic device 301.

According to an embodiment, the electronic device 201 may obtain state information of a battery of the wearable electronic device 301 from the wearable electronic device 301, and display the state information of the battery of the wearable electronic device 301 on the display 260. In this case, the electronic device 201 may further display the state information of the battery of the electronic device 201 according to a user input. For example, the state information of the battery may include information indicating that the battery is being charged and information on the remaining amount of the battery.

According to an embodiment, the electronic device 201 may obtain biometric information of the user who wears the wearable electronic device 301. According to an embodiment, the electronic device 201 may display biometric information of the user on the display 260. For example, the biometric information may include information on a heart rate. According to an embodiment, the electronic device 201 may acoustically provide the biometric information through the speaker 212.

In addition, the electronic device 201 may display, on the display 260, at least one object for causing at least one function related to the wearable electronic device 301 to be executed. According to an embodiment, when an input to the at least one object is identified, the electronic device 201 may transmit, to the wearable electronic device 301, a control signal for causing the at least one function to be executed. According to an embodiment, the electronic device 201 may transmit, based on a voice signal obtained through the microphone 211, a control signal for causing the at least one function to be executed to the wearable electronic device 301. According to an embodiment, the wearable electronic device 301 may execute, based on the control signal, the at least one function. For example, the at least one function may include a function of turning on or off power of the wearable electronic device 301, a function of initializing the wearable electronic device 301, a function of communicatively connecting the wearable electronic device 301 to an external electronic device different from the electronic device 201, a function of updating software of the wearable electronic device 301, or a function of switching the mode of the wearable electronic device 301 to an airplane mode.

FIG. 3 is a block diagram illustrating a system including an electronic device and a wearable electronic device according to an embodiment.

Referring to FIG. 3, according to an embodiment, a system 300 may include an electronic device 201 and a wearable electronic device 301.

According to an embodiment, the electronic device 201 may be implemented as a cradle, and the wearable electronic device 301 may be implemented as a ring-shaped wearable electronic device which can be mounted in the electronic device 201. However, this is a mere example, and the electronic device 201 and the wearable electronic device 301 may be implemented as various types of electronic devices.

According to an embodiment, the electronic device 201 may include a microphone 211, a speaker 212, a processor (e.g., including processing circuitry) 220, a first battery 230, charging circuitry 240, a display 260, and communication circuitry 290. According to an embodiment, the processor 220 may include various processing circuitry and control an overall operation of the electronic device 201. For example, the processor 220 may be implemented equal to or similar to the processor 120 of FIG. 1.

According to an embodiment, the wearable electronic device 301 may include a sensor 310, a processor (e.g., including processing circuitry) 320, a second battery 330, and communication circuitry 390. According to an embodiment, the processor 320 may control an overall operation of the wearable electronic device 301. For example, the processor 320 may be implemented equal to or similar to the processor 120 of FIG. 1.

According to an embodiment, when the wearable electronic device 301 is identified to be mounted in the electronic device 201, the processor 220 may wirelessly transmit power to the wearable electronic device 301 using the charging circuitry 240.

According to an embodiment, the processor 220 may obtain second state information of a second battery 330 through the communication circuitry 290 while wirelessly transmitting the power. In this case, according to an embodiment, the processor 220 may obtain first state information of the first battery 230. For example, the first state information may include information a remaining amount of the first battery 230, information on an available time of the first battery 230, and information on a full-charge time of the first battery 230. For example, the second state information may include information on a remaining amount of the second battery 330, information on an available time of the second battery 330, and information on a full-charge time of the second battery 330.

According to an embodiment, the processor 220 may identify whether the wearable electronic device 301 is in a power on state. According to an embodiment, the processor 220 may identify whether the wearable electronic device 301 is in an on state when a communication signal is received from the wearable electronic device 301 through the communication circuitry 290. According to an embodiment, when the communication signal is not received from the wearable electronic device 301 through the communication circuit 290, the processor 220 may identify that the wearable electronic device 301 is in an off state.

According to an embodiment, when the power on state of the wearable electronic device 301 is identified, the processor 220 may display at least one of the first state information or the second state information. According to an embodiment, the processor 220 may display the second state information on the display 260, or may display the first state information and the second state information on the display 260. According to an embodiment, displaying the second state information on the display 260, or displaying the first state information and the second state information on the display 260 may be configured by the user, or automatically configured by the processor 220. According to the implementation, the processor 220 may also display only the first state information on the display 260.

According to an embodiment, when the power on state of the wearable electronic device 301 is identified, the processor 220 may obtain, from the wearable electronic device 301, biometric information obtained before the wearable electronic device 301 is mounted in the electronic device 201, and display the obtained biometric information on the display 260. According to an embodiment, the biometric information obtained before the wearable electronic device 301 is mounted in the electronic device 201 may include biometric information obtained in the state in which the wearable electronic device 301 is worn on the body part of the user. For example, a sensor 310 may include a photoplethysmography (PPG) sensor or a sensor other than the PPG sensor, which senses various biometric information. For example, the sensor 310 may obtain the PPG signal of the user, a galvanic skin reflex (GSR) signal, a bioelectrical impedance analysis (BIA) signal, and/or an electrocardiogram (ECG) signal. According to an embodiment, the processor 320 may obtain, based on a biometric signal, biometric information including at least one among information on a heart rate, ECG, body fat, a skin temperature, a fertile window, sleeping hours, activity hours, or consumed calories, but may not be limited to the above examples.

According to an embodiment, when the power on state of the wearable electronic device 301 is identified, the processor 220 may also obtain, from the wearable electronic device 301, information on the number steps, obtained before the wearable electronic device 301 is mounted in the electronic device 201. According to an embodiment, information on the number of steps, obtained before the wearable electronic device 301 is mounted in the electronic device 201 may include information on the number of steps, obtained in the state in which the wearable electronic device 301 is worn on the body part of the user. According to an embodiment, when the power on state of the wearable electronic device 301 is identified, the processor 220 may display, on the display 260, information on the number of steps, obtained before the wearable electronic device 301 is mounted in the electronic device 201.

According to an embodiment, the processor 220 may display, based on the identification that the wearable electronic device 301 is in the power off state, an object capable of executing a function of turning on the power of the wearable electronic device 301, on the display 260. According to an embodiment, when a user input to the object capable of executing the function of turning on the power of the wearable electronic device 301, the processor 220 may transmit, to the wearable electronic device 301, a control signal for causing the power of the wearable electronic device 301 to be turned on. According to an embodiment, the processor 320 may turn on, based on the acquisition of the control signal for causing the power of the wearable electronic device 301 to be turned on, the power of the wearable electronic device 301.

According to an embodiment, when the power of the wearable electronic device 301 is identified to be turned on, the processor 220 may display, on the display 260, at least one object corresponding to at least one function related to the wearable electronic device 301. For example, the at least one function may include a function of turning on or off power of the wearable electronic device 301, a function of initializing the wearable electronic device 301, a function of connecting the wearable electronic device 301 to an external electronic device different from the wearable electronic device 301, a function of updating software of the wearable electronic device 301, or a function of switching the mode of the wearable electronic device 301 to an airplane mode. For example, the function of initializing the wearable electronic device 301 may include a factory initialization function. For example, the external electronic device may include a wearable device which can be worn on the wrist, or a smartphone.

According to an embodiment, when an input to a first object among the at least one object is identified, the processor 220 may transmit a control signal for causing the wearable electronic device 301 to execute a first function corresponding to the first object to the wearable electronic device 301 through the communication circuitry 290. For example, the input to the first object may include a touch input, a tap input, or a long press input to the first object. For example, the processor 220 may also obtain a voice signal for causing the first function corresponding to the first object to be executed through the microphone 211. According to an embodiment, the processor 220 may analyze the voice signal, and transmit a control signal for causing a function corresponding to the voice signal to be executed to the wearable electronic device 301. According to an embodiment, the wearable electronic device 301 may execute, based on the control signal, the first function.

According to an embodiment, when the voice signal or the input to the first object capable of executing a function of initializing the wearable electronic device 301 is obtained, the processor 220 may store, in memory (not shown) included in the electronic device 201, biometric information according to a user input. According to an embodiment, the processor 220 may transmit the control signal for causing the wearable electronic device 301 to be initialized to the wearable electronic device 301 after storing the biometric information in the memory (not shown) included in the electronic device 201 according to the user input.

According to an embodiment, when the second battery 330 is identified to be fully charged, the processor 220 may output, using the display 260 or the speaker 212, information indicating that the second battery 330 has been fully charged.

According to an embodiment, even when the wearable electronic device 301 is identified not to be mounted in the electronic device 201, the processor 220 may display the second state information of the second battery 330 on the display 260. For example, the second state information may include information on a remaining amount of the second battery 330 and information on an available time of the second battery 330.

According to an embodiment, when the remaining amount of the second battery 330 is identified to be less than a specific value, the processor 220 may transmit, to the wearable electronic device 301, a control signal for causing information on a notification for charging the second battery 330 to be output. For example, the specified value may indicate a value for determining whether to transmit the control signal. For example, the specified value may be configured by the user, or may be automatically configured by the processor 220.

According to an embodiment, the processor 220 may obtain biometric information of the user from the wearable electronic device 301 through the communication circuitry 290 in the state in which the wearable electronic device 301 is not mounted in the electronic device 201. According to an embodiment, the processor 220 may display the biometric information of the user in which the wearable electronic device 301 is not mounted in the electronic device 201.

According to an embodiment, the processor 220 may obtain the number of steps of the user from the wearable electronic device 301. According to an embodiment, the processor 220 may obtain, based on the number of steps and a heart rate included in the biometric information, whether the user is working out. For example, when the heart rate is identified to be higher than a first pre-configured value and the number of steps is identified to be greater than a second pre-configured value, the processor 220 may identify that the user is working out. For example, the first pre-configured value and the second pre-configured value may indicate values for determining whether the user is working out. For example, the first pre-configured value and the second pre-configured value may be configured by the user, or may be automatically configured by the processor 220. For example, the first pre-configured value may include a value different from the second pre-configured value.

According to an embodiment, when the identification that the user is working out is made, the processor 220 may transmit, to the wearable electronic device 301, a control signal for causing the wearable electronic device 301 to be configured in a workout mode.

According to an embodiment, when the heart rate value obtained from the wearable electronic device 301 corresponds to a pre-configured heart rate value, the processor 220 may output, based on the wearable electronic device 301 having been configured to be in the workout mode, notification information related to the heart rate value through the display 260 or the speaker 212.

According to an embodiment, the processor 220 may identify, based on the wearable electronic device 301 being configured to be in the workout mode, whether the heart rate value obtained from the wearable electronic device 301 is included in a heart rate interval corresponding to an aerobic workout or a heart rate interval corresponding to an anaerobic workout. According to an embodiment, when the heart rate value is identified to be included in the heart rate interval corresponding to an aerobic workout, the processor 220 may output, through the display 260 or the speaker 212, information on a notification for maintaining, for a pre-configured time (e.g., 10 minutes), a workout speed at a time point at which the heart rate value is obtained. According to an embodiment, when the heart rate value is identified to be included in the heart rate interval corresponding to an anaerobic workout, the processor 220 may output, through the display 260 or the speaker 212, information on a notification for maintaining, for a pre-configured time (e.g., five minutes), a workout speed at a time point at which the heart rate value is obtained.

According to an embodiment, when identification that the workout has been performed by the user for a pre-configured time is made based on the wearable electronic device 301 being configured in the workout mode, the processor 220 may output notification information related to a workout time through the display 260 or the speaker 212. According to an embodiment, when identification that the workout has been performed by the user for a pre-configured time, the processor 220 may display, on the display 260, a pop-up window for allowing the user to select whether to terminate the workout mode.

According to an embodiment, based on the wearable electronic device 301 being configured in the workout mode, the processor 220 may output, through the display 260 or the speaker 212, notification information related to an average speed of the user according to an acceleration value obtained from the wearable electronic device 301.

According to an embodiment, the processor 220 may execute, based on the wearable electronic device 301 being configured in the workout mode, an always on display (AOD) mode of the display 260.

According to an embodiment, the processor 220 may identify, based on the wearable electronic device 301 being configured in the workout mode, whether a remaining amount of the second battery 330 is greater than a specific value. According to an embodiment, when the remaining amount of the second battery 330 is identified to be greater than the specific value, the processor 220 may identify the quality of a biometric signal obtained by the wearable electronic device 301. For example, the specific value may include a value for determining whether to identify the quality of the biometric signal. For example, the quality of the biometric signal may include a value (e.g., signal to noise ratio (SNR)) related to a noise signal ratio compared to a biometric signal.

According to an embodiment, when the quality of the biometric signal is lower than a specific quality, the processor 220 may transmit, to the wearable electronic device 301, a control signal for causing a measurement period of a sensor 310 to be adjusted to a second period shorter than a pre-configured first period. For example, the specific quality may include a value for determining whether to transmit the control signal for causing the measurement period of the sensor 310 to be adjusted. According to an embodiment, when the quality of the biometric signal is lower than the specific value, the processor 220 may also transmit, to the wearable electronic device 301, a control signal for causing the wearable electronic device 301 to output notification information of the wearing state of the wearable electronic device 301.

According to an embodiment, the processor 220 may obtain biometric information and acceleration information of the user from the wearable electronic device 301 through the communication circuit 290 in the state in which the wearable electronic device 301 is not mounted in the electronic device 201. According to an embodiment, the processor 220 may identify, based on the heart rate and acceleration values, whether the user is sleeping.

According to an embodiment, when the identification that the user is sleeping is made, the processor 220 may identify whether the remaining amount of the second battery 330 is less than a specified value. For example, the specific value may include a value for determining whether to transmit a control signal for causing the wearable electronic device 301 to be configured to be in a power saving mode. According to an embodiment, when the remaining amount of the second battery 330 is identified to be less than the specific value, the processor 220 may transmit, to the wearable electronic device 301, a control signal for causing the wearable electronic device 301 to be configured in a power saving mode. For example, when the wearable electronic device 301 is configured to be in the power saving mode, at least a part of the biometric information of the user may not be obtained. For example, when being configured to be in the power saving mode, the wearable electronic device 301 may obtain information on the heart rate, skin temperature, and fertile window, and may not obtain information on the oxygen saturation, sleep apnea, and snoring.

According to an embodiment, when the remaining amount of the second battery 330 is identified not to be less than a specific value, the processor 220 may transmit, to the wearable electronic device 301, a control signal for causing the wearable electronic device 301 to be in a sleep mode.

According to an embodiment, when the wearable electronic device 301 is configured to be in a sleep mode, the processor 220 may obtain information on the heart rate, skin temperature, and fertile window, oxygen saturation, sleep apnea, and snoring.

According to an embodiment, the processor 220 may analyze a voice signal obtained through the microphone 211, and may output a result analyzed through the speaker 212. For example, the voice signal may include a signal related to state information of the second battery 330 and a signal related to state information of the first battery 230.

According to an embodiment, according to a user input, the processor 220 may charge the wearable electronic device 301 in a high-speed charging mode or a normal charging mode.

According to an embodiment, information displayed through the display 260 when the wearable electronic device 301 is mounted in the electronic device 201 may be different from information displayed through the display 260 when the wearable electronic device 301 is worn on the body part of the user. According to an embodiment, substantially the same operations of the electronic device 201, performed when the wearable electronic device 301 is mounted in the electronic device 201, may be also applied to a case where the wearable electronic device 301 is mounted in the electronic device 201. According to an embodiment, substantially the same operations of the electronic device 201, performed when the wearable electronic device 301 is not mounted in the electronic device 201, may be also applied to a case where the wearable electronic device 301 is mounted in the electronic device 201.

The operations of the electronic device 201, described below with reference to the drawings, may be performed by the processor 220. However, for convenience of description, the operations performed by the processor 220 will be described to be operated by the electronic device 201. The operations of the wearable electronic device 301, described below with reference to the drawings, may be performed by the processor 320. However, for convenience of description, the operations performed by the processor 320 will be described to be operated by the wearable electronic device 301.

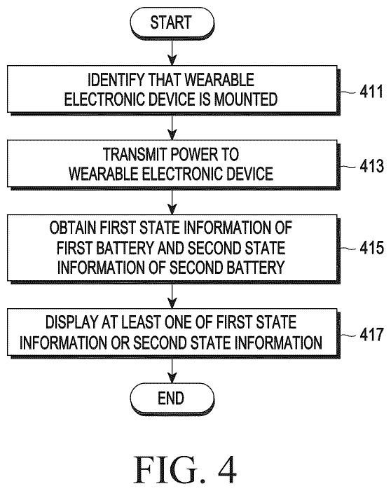

FIG. 4 is a flow chart illustrating an operation of displaying state information of a battery by an electronic device according to an embodiment.

In the embodiments below, the respective operations may be sequentially performed, but are not necessarily performed sequentially. For example, the orders of the respective operations may be changed, or at least two operations may be performed in parallel.

Referring to FIG. 4, according to an embodiment, in operation 411, an electronic device 201 (e.g., the electronic device 201 of FIG. 3) may identify that a wearable electronic device 301 (e.g., the wearable electronic device 301 of FIG. 3) is mounted in the electronic device 201.

According to an embodiment, in operation 413, the electronic device 201 may wirelessly transmit power to the wearable electronic device 301 using charging circuitry 240 (e.g., the charging circuitry 240 of FIG. 3).

According to an embodiment, in operation 415, the electronic device 201 may obtain first state information of a first battery (e.g., the first battery 230 of FIG. 3) included in the electronic device 201 and second state information of a second battery 330 (e.g., the second battery 330 of FIG. 3) included in the wearable electronic device 301. For example, the first state information may include information on a remaining amount of the first battery 230, information on an available time of the first battery 230, and information on a full-charge time of the first battery 230. For example, the second state information may include information on a remaining amount of the second battery 330, information on an available time of the second battery 330, and information on a full-charge time of the second battery 330.

According to an embodiment, in operation 417, the electronic device 201 may display at least one of the first state information or the second state information on a display 260 (e.g., the display 260 of FIG. 3).

According to an embodiment, the electronic device 201 may identify whether the wearable electronic device 301 is in a power on state. According to an embodiment, when a communication signal is received from the wearable electronic device 301 through communication circuitry 290 (e.g., the communication circuitry 290 of FIG. 3), the electronic device 201 may identify that the wearable electronic device 301 is in an on state. According to an embodiment, when the communication signal is not received from the wearable electronic device 301 through the communication circuitry 290 (e.g., the communication circuitry 290 of FIG. 3), the electronic device 201 may identify that the wearable electronic device 301 is in an off state.

According to an embodiment, the electronic device 201 may display, based on the identification that the wearable electronic device 301 is in the power on state, at least one of the first state information or the second state information. According to an embodiment, the electronic device 201 may display the second state information on the display 260, or may display the first state information and the second state information on the display 260.

According to an embodiment, based on the identification that the wearable electronic device 301 is in a power off state, the electronic device 201 may display, on the display 260, a function of turning on the power of the wearable electronic device 301. According to an embodiment, when a user input to an object capable of executing the function of turning on the power of the wearable electronic device 301 is identified, the electronic device 201 may transmit, to the wearable electronic device 301, a control signal for turning on the power of the wearable electronic device 301. According to an embodiment, the wearable electronic device 301 may turn on, based on the obtaining of the control signal for turning on the power of the wearable electronic device 301, the power of the wearable electronic device 301.

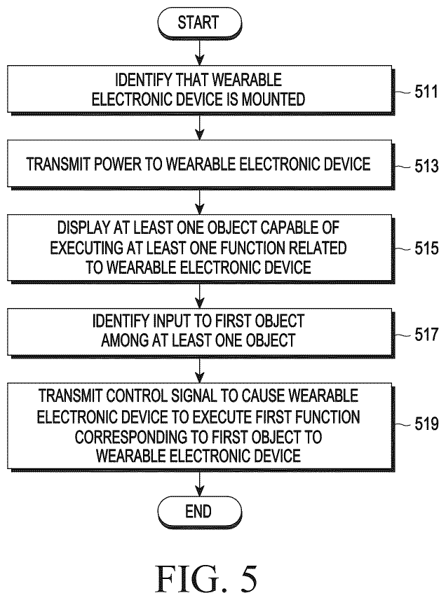

FIG. 5 is a flow chart illustrating an operation of transmitting a control signal to a wearable electronic device by an electronic device to execute a function of the wearable electronic device according to an embodiment.

In the embodiments below, the respective operations may be sequentially performed, but are not necessarily performed sequentially. For example, the orders of the respective operations may be changed, or at least two operations may be performed in parallel.

Referring to FIG. 5, according to an embodiment, in operation 511, an electronic device 201 (e.g., the electronic device 201 of FIG. 3) may identify that a wearable electronic device 301 (e.g., the wearable electronic device 301 of FIG. 3) is mounted in the electronic device 201.

According to an embodiment, in operation 513, the electronic device 201 may wirelessly transmit, based on the identification that the wearable electronic device 301 is mounted in the electronic device 201, power to the wearable electronic device 301 using charging circuitry 240 (e.g., the charging circuitry 240 of FIG. 3).

According to an embodiment, in operation 515, the electronic device 201 may display, on a display 260 (e.g., the display 260 of FIG. 3), at least one object capable of executing at least one function related to the wearable electronic device 301 while wirelessly transmitting power to the wearable electronic device 301. For example, the at least one function may include a function of turning on or off power of the wearable electronic device 301, a function of initializing the wearable electronic device 301, a function of communicatively connecting the wearable electronic device 301 to an external electronic device different from the electronic device 201, a function of updating software of the wearable electronic device 301, or a function of switching the mode of the wearable electronic device 301 to an airplane mode.

According to an embodiment, in operation 517, the electronic device 201 may identify an input to a first object among the at least one object. For example, the input to the first object may include a touch input, a tap input, or a long press input to the first object. For example, the electronic device 201 may also obtain a voice signal for causing a first function corresponding to the first object to be executed through a microphone 211 (e.g., the microphone 211 of FIG. 3).

According to an embodiment, in operation 519, when a first input is identified, the electronic device 201 may transmit, to the wearable electronic device 301 through communication circuitry 290 (e.g., the communication circuitry 290 of FIG. 3), a control signal to cause the wearable electronic device 301 to execute the first function corresponding to the first object. According to an embodiment, the electronic device 201 may analyze a voice signal and also transmit, based on a result of the analysis, a control signal for executing the first function to the wearable electronic device 301. According to an embodiment, the wearable electronic device 301 may execute, based on the control signal obtained from the electronic device 201, the first function.

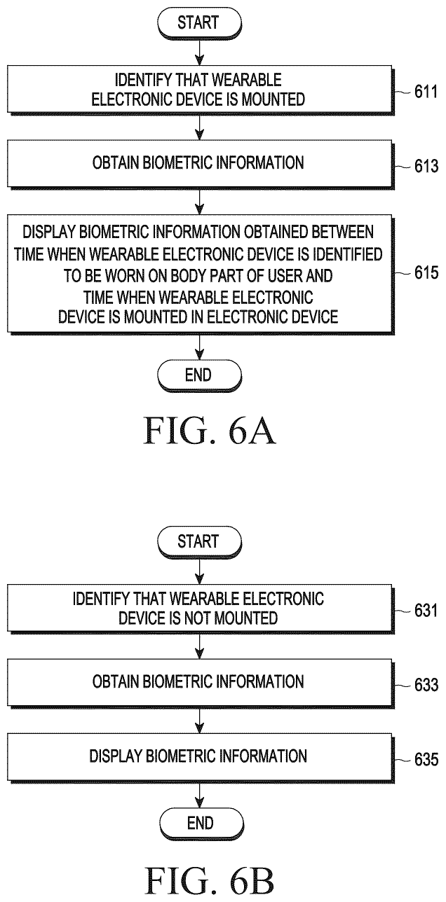

FIG. 6A is a flowchart illustrating an operation of displaying biometric information by an electronic device when a wearable electronic device is mounted in the electronic device according to an embodiment.

In the embodiments below, the respective operations may be sequentially performed, but are not necessarily performed sequentially. For example, the orders of the respective operations may be changed, or at least two operations may be performed in parallel.

Referring to FIG. 6A, according to an embodiment, in operation 611, an electronic device 201 (e.g., the electronic device 201 of FIG. 3) may identify that a wearable electronic device 301 (e.g., the wearable electronic device 301 of FIG. 3) is mounted in the electronic device 201.

According to an embodiment, in operation 613, the electronic device 201 may obtain, from the wearable electronic device 301, biometric information of a user, obtained by the wearable electronic device 301 in a time interval between a time point at which identification that the wearable electronic device is worn on the body part of the user is made and a time point at which identification that the wearable electronic device 301 is mounted in the electronic device 201 is made.

According to an embodiment, the biometric information of the user may include biometric information obtained by a sensor 310 (e.g., the sensor 310 of FIG. 3) before the wearable electronic device 301 is mounted in the electronic device 201. According to an embodiment, the biometric information may include at least one among information on a heart rate, ECG, body fat, a skin temperature, a fertile window, sleeping hours, activity hours, pressure, or consumed calories, but may not be limited to the above examples. According to an embodiment, the biometric information of the user may include biometric information of the user, obtained by the wearable electronic device 301 at a specific time point included in a time interval between a time point at which identification that the wearable electronic device is worn on the body part of the user is made and a time point at which identification that the wearable electronic device 301 is mounted in the electronic device 201 is made. For example, the specific time point may be configured by the user, or may be automatically configured by the wearable electronic device 301 or the electronic device 201.

According to an embodiment, the electronic device 201 may also obtain, from the wearable electronic device 301, average sensing values (e.g., an average heart rate, an average ECG, an average skin temperature, etc.) related to biometric information obtained in a time interval between a time point at which identification that the wearable electronic device is worn on the body part of the user is made and a time point at which the wearable electronic device 301 is mounted in the electronic device 201 is made.

According to an embodiment, the electronic device 201 may also obtain, from the wearable electronic device 301, information on the number of steps, obtained in a time interval between a time point at which identification that the wearable electronic device 301 is worn on the body part of the user is made and a time point at which identification that the wearable electronic device 301 is mounted in the electronic device 201 is made.

According to an embodiment, in operation 615, the electronic device 201 may display, on a display 260 (e.g., the display 260 of FIG. 3), the biometric information obtained between a time when the wearable electronic device 301 is identified to be worn by the user and a time when the wearable electronic device 301 is identified to be mounted in the electronic device 201. According to an embodiment, the electronic device 201 may display, on the display 260, information on the number of steps.

For example, the electronic device 201 may display, on the display 260, information on an average walking speed of the user, obtained in a time interval between a time point at which identification that the wearable electronic device 301 is worn on the body part of the user is made and a time point at which identification that the wearable electronic device 301 is mounted in the electronic device 201 is made.

For example, the electronic device 201 may display, on the display 260, information on at least one of an average heart rate, a maximum heart rate, a minimum hear rate, an average ECG, a maximum blood pressure, a minimum blood pressure, or an average skin temperature, obtained in a time interval between a time point at which identification that the wearable electronic device 301 is worn on the body part of the user is made and a time point at which identification that the wearable electronic device 301 is mounted in the electronic device 201 is made.

For example, the electronic device 201 may display, on the display 260, biometric information of the user, obtained by the wearable electronic device 301 at a specific time point included in a time interval between a time point at which identification that the wearable electronic device 301 is worn on the body part of the user is made and a time point at which identification that the wearable electronic device 301 is mounted in the electronic device 201 is made.

However, this is simply an example, and in the embodiments of the disclosure, information displayed by the electronic device 201 may not be limited thereto.

FIG. 6B is a flowchart illustrating an operation of displaying biometric information by an electronic device when a wearable electronic device is not mounted in the electronic device according to an embodiment.

In the embodiments below, the respective operations may be sequentially performed, but are not necessarily performed sequentially. For example, the orders of the respective operations may be changed, or at least two operations may be performed in parallel.

Referring to FIG. 6B, according to an embodiment, in operation 631, an electronic device 201 (e.g., the electronic device 201 of FIG. 3) may identify that a wearable electronic device 301 (e.g., the wearable electronic device 301 of FIG. 3) is not mounted in the electronic device 201.

According to an embodiment, in operation 633, the electronic device 201 may obtain biometric information of a user from the wearable electronic device 301 through communication circuitry 290 (e.g., the communication circuitry 290 of FIG. 3). According to an embodiment, the biometric information of the user may include information on at least one of a heart rate, ECG, body fat, a skin temperature, a fertile window, sleeping hours, activity hours, pressure, or consumed calories.

According to an embodiment, the electronic device 201 may obtain the biometric information of the user from the wearable electronic device 301 at each specific time. For example, the specific time may be configured by the user, or may be configured by the electronic device 201 or the wearable electronic device 301. According to an embodiment, the electronic device 301 may obtain the biometric information of the user from the wearable electronic device 301 in real time.

According to an embodiment, in operation 633, the electronic device 201 may display the biometric information obtained from the wearable electronic device 301 through a display 260 (e.g., the display 260 of FIG. 2).

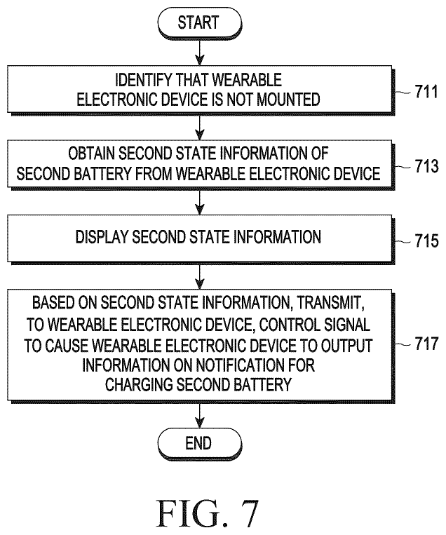

FIG. 7 is a flowchart illustrating an operation of transmitting, to a wearable electronic device by an electronic device, a control signal for causing information on a notification for charging a second battery of the wearable electronic device to be output according to an embodiment.

In the embodiments below, the respective operations may be sequentially performed, but are not necessarily performed sequentially. For example, the orders of the respective operations may be changed, or at least two operations may be performed in parallel.

Referring to FIG. 7, according to an embodiment, in operation 711, an electronic device 201 (e.g., the electronic device 201 of FIG. 3) may identify that a wearable electronic device 301 (e.g., the wearable electronic device 301 of FIG. 3) is not mounted in the electronic device 201.

According to an embodiment, in operation 713, the electronic device 201 may obtain second state information of a second battery 330 (e.g., the second battery 330 of FIG. 3) from the wearable electronic device 301 through communication circuitry 290 (e.g., the communication circuitry 290 of FIG. 3). For example, the second state information may include information on a remaining amount of the second battery 330 and information on an available time of the second battery 330.

According to an embodiment, in operation 715, the electronic device 201 may display the second state information on a display 260 (e.g., the display 260 of FIG. 3).

According to an embodiment, in operation 717, based on the second state information, the electronic device 201 may transmit, to the wearable electronic device 301, a control signal to cause output information on a notification for charging the second battery 330.

According to an embodiment, when the remaining amount of the second battery 330 is identified to be less than a specific value, based on the second state information the electronic device 201 may transmit, to the wearable electronic device 301, a control signal for causing information on a notification for charging the second battery 330 to be output. For example, the specific value may include a value determined to be transmitted to the wearable electronic device 301. According to an embodiment, the wearable electronic device 301 may visually or acoustically output, based on obtaining of the control signal for causing the information on the notification for charging the second battery 330 to be output, the notification information.

According to an embodiment, when the remaining amount of the second battery 330 is identified to be not less than the specified value, based on the second state information, the electronic device 201 may not transmit, to the wearable electronic device 301, the control signal for causing the information on the notification for charging the second battery 330 to be output.

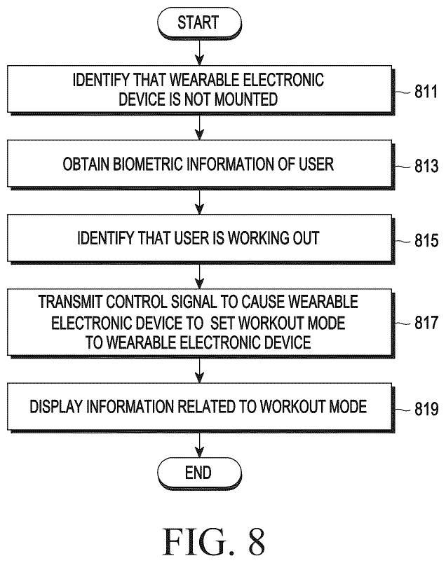

FIG. 8 is a flowchart illustrating an operation of identifying, by an electronic device, whether a user wearing a wearable electronic device is working out according to an embodiment.

In the embodiments below, the respective operations may be sequentially performed, but are not necessarily performed sequentially. For example, the orders of the respective operations may be changed, or at least two operations may be performed in parallel.

Referring to FIG. 8, according to an embodiment, in operation 811, an electronic device 201 (e.g., the electronic device 201 of FIG. 3) may identify that a wearable electronic device 301 (e.g., the wearable electronic device 301 of FIG. 3) is not mounted in the electronic device 201.

According to an embodiment, in operation 813, the electronic device 201 may obtain biometric information of a user from the wearable electronic device 301 through communication circuitry 290 (e.g., the communication circuitry 290 of FIG. 3). According to an embodiment, the biometric information may include information on at least one of a heart rate, ECG, body fat, the number of steps, a skin temperature, a fertile window, sleeping hours, activity hours, or consumed calories. According to an embodiment, the electronic device 201 may obtain acceleration information from the wearable electronic device 301 through the communication circuitry 290.

According to an embodiment, in operation 815, the electronic device 201 may identify, based on the biometric information and the number of steps, that the user is working out. For example, the electronic device 201 may identify, based on the heart rate included in the biometric information and the number of steps, whether the user is working out. For example, when the heart rate is identified to be greater than a first pre-configured value and the number of steps is identified to be greater than a second pre-configured value, the electronic device 201 may identify that the user is working out. For example, the first pre-configured value and the second pre-configured value may indicate a value for determining whether the user is working out.

According to an embodiment, in operation 817, when identification that the user is working out is made, the electronic device 201 may transmit, to the wearable electronic device 301, a control signal to cause the wearable electronic device 301 to set in a workout mode.

According to an embodiment, when the wearable electronic device 301 is configured to be in the workout mode, biometric information of the user, information on the number of steps of the user, information on a workout time, or information of an average speed may be output through a visual or acoustic means.

According to an embodiment, in operation 819, the electronic device 201 may display, based on the identification that the user is working out, information related to the workout mode through a display 260 (e.g., the display 260 of FIG. 2).

According to an embodiment, the electronic device 201 may display, through the display 260, the biometric information, information on the number of steps of the user, information on the workout time, or information on the average speed, which are obtained from the wearable electronic device 301 at each specified time or in real time.

According to an embodiment, when the heart rate value obtained from the wearable electronic device 301 corresponds to a pre-configured heart rate value, the electronic device 201 may display notification information related to the heart rate value through the display 260 or a speaker 212 (e.g., the speaker 212 of FIG. 3).

According to an embodiment, the electronic device 201 may identify whether the heart rate value obtained from the wearable electronic device 301 is included in a heart rate interval corresponding to an aerobic workout or a heart rate interval corresponding to an anaerobic workout. According to an embodiment, when the heart rate value is identified to be included in the heart rate interval corresponding to the aerobic workout, the electronic device 201 may output, through the display 260 or the speaker 212, information on a notification for maintaining, for a pre-configured time (e.g., 10 minutes), a workout speed at a time point at which the heart rate value is obtained. According to an embodiment, when the heart rate value is identified to be included in the heart rate interval corresponding to the anaerobic workout, the electronic device 201 may output, through the display 260 or the speaker 212, information on a notification for maintaining, for a pre-configured time (e.g., five minutes), a workout speed at a time point at which the heart rate value is obtained.

According to an embodiment, when identification that the workout has been performed for a pre-configured time is made, the electronic device 201 may output notification information related to the workout time through the display 260 or the speaker 212.

According to an embodiment, the electronic device 201 may output, through the display 260 or the speaker 212, notification information related to an average speed of the user according to an acceleration value obtained from the wearable electronic deice 301.

According to an embodiment, the electronic device 201 may execute an always on display (AOD) function of the display 260.

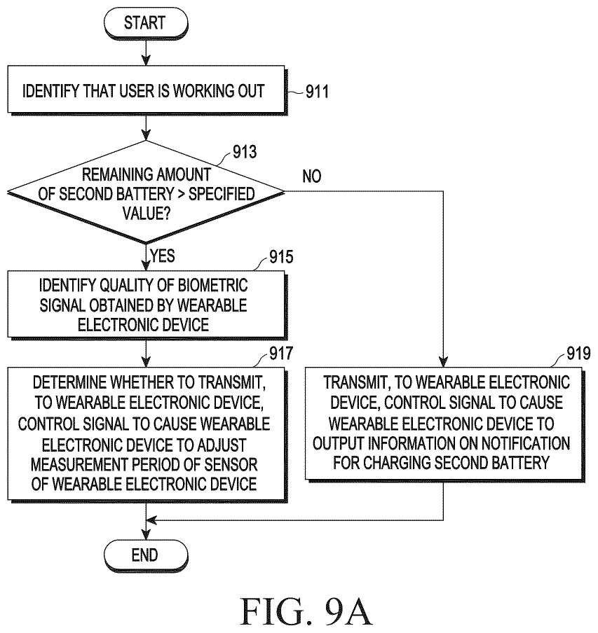

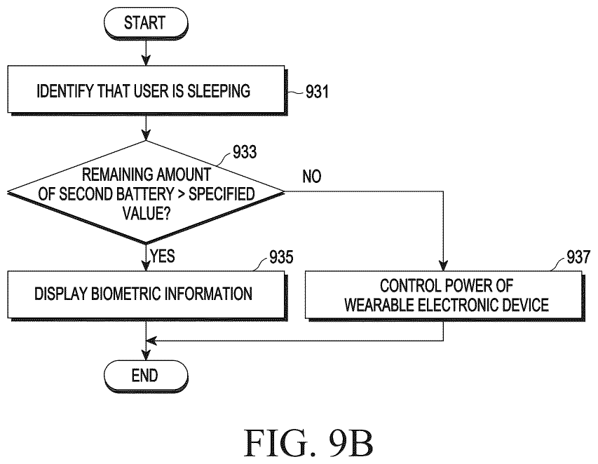

FIG. 9A is a flowchart illustrating an operation of transmitting, by an electronic device, a control signal for causing a measurement period of a sensor of a wearable electronic device to be adjusted according to an embodiment.

In the embodiments below, the respective operations may be sequentially performed, but are not necessarily performed sequentially. For example, the orders of the respective operations may be changed, or at least two operations may be performed in parallel.

Referring to FIG. 9A, according to an embodiment, in operation 911, an electronic device 201 (e.g., the electronic device 201 of FIG. 3) may identify that a user wearing a wearable electronic device 301 (e.g., the wearable electronic device 301 of FIG. 3) is working out.

According to an embodiment, in operation 913, the electronic device 201 may identify whether a remaining amount of a second battery 330 (e.g., the second battery 330 of FIG. 3) of the wearable electronic device 301 is greater than a specific value.

According to an embodiment, when the remaining amount of the second battery 330 is identified to be greater than the specified value (Yes in operation 913), the electronic device 201 may identify, in operation 915, the quality of a biometric signal obtained by the wearable electronic device 301. For example, the specified value may include a value for determining whether to identify the quality of the biometric signal.

According to an embodiment, in operation 917, the electronic device 201 may determine, based on the quality of the biometric signal, whether to transmit a control signal to cause wearable electronic device adjust a measurement period of a sensor 310 (e.g., the sensor 310 of FIG. 3) of the wearable electronic device 301 to the wearable electronic device 301. For example, the quality of the biometric signal may include a value (e.g., signal to noise ratio (SNR)) related to a noise signal compared to a biometric signal.

According to an embodiment, when the quality of the biometric signal is lower than a specified quality, the electronic device 201 may transmit the control signal for causing the measurement period of the sensor 310 to be adjusted to the wearable electronic device 301. For example, the specified quality may include a value for determining whether to transmit the control signal for causing the measurement period of the sensor 310 to be adjusted. For example, the electronic device 201 may transmit the control signal for adjusting the measurement period of the sensor 310 to a second period shorter than a pre-configured first period to the wearable electronic device 301. According to the implementation, when the quality of the biometric signal is lower than the specified quality, the electronic device 201 may transmit a control signal for causing the measurement period of the sensor 310 to be adjusted to a third period longer than the pre-configured first period to the wearable electronic deice 301.

According to the implementation, according to an embodiment, when the quality of the biometric signal is higher than the specified quality, the electronic device 201 may transmit a control signal for causing the measurement period of the sensor 310 to be adjusted to the wearable electronic device 301. For example, the electronic device 201 may transmit the control signal for causing the measurement period of the sensor 310 to be adjusted to a third period longer than the pre-configured first period to the wearable electronic device 301. According to the implementation, when the quality of the biometric signal is higher than the specified quality, the electronic device 201 may transmit a control signal for causing the measurement period of the sensor 310 to be adjusted to a second period shorter than the pre-configured first period to the wearable electronic device 301.

According to an embodiment, when the remaining amount of the second battery 330 is identified not to be greater than the specified value (No in operation 913), the electronic device 201 may transmit, to the wearable electronic device 301, a control signal to cause wearable electronic device output information on a notification for charging the second battery in operation 919.

According to an embodiment, when the remaining amount of the second battery 330 is identified not to be greater than the specified value, the wearable electronic device 301 may not transmit biometric information of the user to the electronic device 201. For example, the biometric information may include information on at least one of a heart rate, ECG, body fat, a skin temperature, a fertile window, sleeping hours, activity hours, blood pressure, or consumed calories. According to an embodiment, when the remaining amount of the second battery 330 is identified not to be greater than the specified value, the wearable electronic device 301 may not transmit at least a part of the biometric information of the user to the electronic device 201.

FIG. 9B is a flowchart illustrating an operation of displaying biometric information of a user by an electronic device when a wearable electronic device is configured to be in a sleep mode according to an embodiment.

In the embodiments below, the respective operations may be sequentially performed, but are not necessarily performed sequentially. For example, the orders of the respective operations may be changed, or at least two operations may be performed in parallel.

Referring to FIG. 9B, according to an embodiment, in operation 931, an electronic device 201 (e.g., the electronic device 201 of FIG. 3) may identify that a user wearing a wearable electronic device 301 (e.g., the wearable electronic device 301 of FIG. 3) is sleeping. For example, the electronic device 201 may identify that the user is sleeping, based on heart rate and acceleration values of the user, obtained from the wearable electronic device 301.

According to an embodiment in operation 933, the electronic device 201 may identify whether a remaining amount of a second battery 330 (e.g., the second battery 330 of FIG. 3) is greater than a specified value.

According to an embodiment, in operation 935, the electronic device 201 may display, through a display 260 (e.g., the display 260 of FIG. 2), biometric information of the user when the remaining amount of the second battery 330 is identified to be greater than the specified value (Yes in operation 933).

According to an embodiment, information transmitted to the electronic device 201 by the wearable electronic device 301 when the user is sleeping may be different from information transmitted to the electronic device 201 by the wearable electronic device 301 when the user is working out. For example, when identification that the user is sleeping is made, the wearable electronic device 301 may transmit information on the heart rate, skin temperature, fertile window, oxygen saturation, sleep apnea, and snoring to the electronic device 201. For example, when identification that the user is working out is made, the wearable electronic device 301 may transmit information on the number of steps of the user, workout hours, heart rate, skin temperature, fertile window, oxygen saturation, sleep apnea, and snoring to the electronic device 201. However, this is a mere example, and an example of the information transmitted to the electronic device 201 may not be limited thereto.

According to an embodiment, the electronic device 201 may obtain the information of the heart rate, skin temperature, fertile window, oxygen saturation, sleep apnea, and snoring from the wearable electronic device 301.

According to an embodiment, when the remaining amount of the second battery 330 is identified not to be greater than the specified value (No in operation 933), the electronic device 201 may control power of the wearable electronic device 301 in operation 937.

According to an embodiment, the electronic device 201 may transmit a control signal for causing the wearable electronic device 301 to be configured in a power saving mode to the wearable electronic device 301. For example, when being configured to be in the power saving mode, the wearable electronic device 301 may obtain the information on the oxygen saturation, sleep apnea, and snoring, and may not obtain the information on the heart rate, skin temperature, and fertile window.

According to an embodiment, the electronic device 201 may transmit a control signal for causing power of the wearable electronic device 301 to be turned off to the wearable electronic device 301. According to an embodiment, the wearable electronic device 301 may turn off, based on the control signal, the power of the wearable electronic device 301.

According to an embodiment, the electronic device 201 may store information on a sleeping state of the user in memory of the electronic device 201. For example, the information on the sleeping state of the user may include information on a time point at which the user starts to sleep, information on a time point at which the user stops sleeping, and information on a total of sleeping hours of the user. According to an embodiment, the electronic device 201 may predict, based on the information on the sleeping state of the user, a sleeping start time (e.g., 10 P.M.).

According to an embodiment, the electronic device 201 may identify the remaining amount of the second battery 330 before a specified time from the predicted sleeping start time of the user. According to an embodiment, based on the remaining amount of the second battery 330 being less than the specified value, the electronic device 201 may transmit, to the wearable electronic device 301, a control signal for causing information on a notification for charging the second battery 330 to be output.

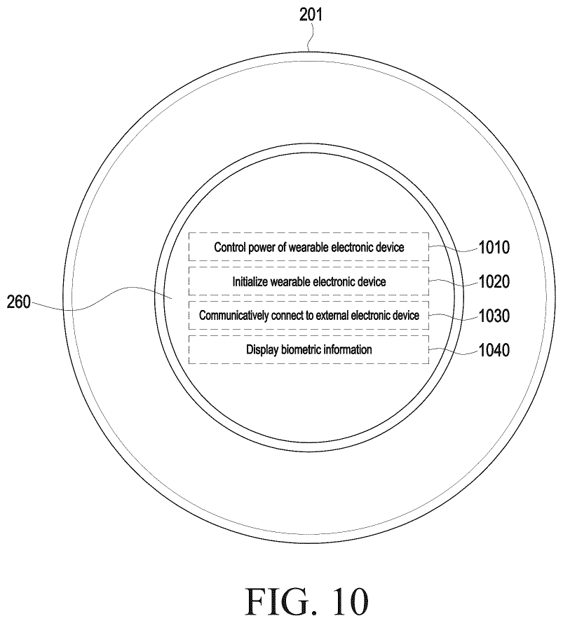

FIG. 10 illustrates a screen of displaying at least one object corresponding to at least one function related to a wearable electronic device on a display by an electronic device according to an embodiment.

Referring to FIG. 10, according to an embodiment, when a wearable electronic device 301 (e.g., the wearable electronic device 301 of FIG. 3) is identified to be mounted, an electronic device 201 (e.g., the electronic device 201 of FIG. 3) may display at least one object corresponding to at least one function related to the wearable electronic device 301 on a display 260 (e.g., the display 260 of FIG. 3). According to an embodiment, even though the wearable electronic device 301 is identified not to be mounted in the electronic device 201, when the electronic device 201 and the wearable electronic device 301 are identified to be connected to each other via short-distance communication, the electronic device 201 may display at least one object corresponding to at least one function related to the wearable electronic device 301 on the display 260.

According to an embodiment, the electronic device 201 may display a first object 1010 corresponding to a function of controlling the power of the wearable electronic device 301 on the display 260. According to an embodiment, the function of controlling the power may include a function of turning on or off the power of the wearable electronic device 301.

According to an embodiment, the electronic device 201 may display a second object 1020 for initializing the wearable electronic device 301 on the display 260. According to an embodiment, when an input to the second object 1020 is identified, the electronic device 201 may determine whether to store biometric information in memory of the electronic device 201 according to a user input. According to an embodiment, after determining whether to store the biometric information in the memory of the electronic device 201 according to the user input, the electronic device 201 may transmit a control signal for causing the wearable electronic device 301 to be initialized to the wearable electronic device 301.

According to an embodiment, the electronic device 201 may display, on the display 260, a third object 1030 corresponding to a function of communicatively connecting the wearable electronic device 301 to an external electronic device different from the electronic device 201. For example, the external electronic device may include a smartphone, a tablet PC, or a wearable electronic device which can be worn on the wrist.

According to an embodiment, the electronic device 201 may display a fourth object 1040 corresponding to a function of displaying biometric information obtained from the wearable electronic device 301 on the display 260. For example, the biometric information may include information on at least one of a heart rate, ECG, body fat, a skin temperature, a fertile window, sleeping hours, activity hours, or consumed calories.

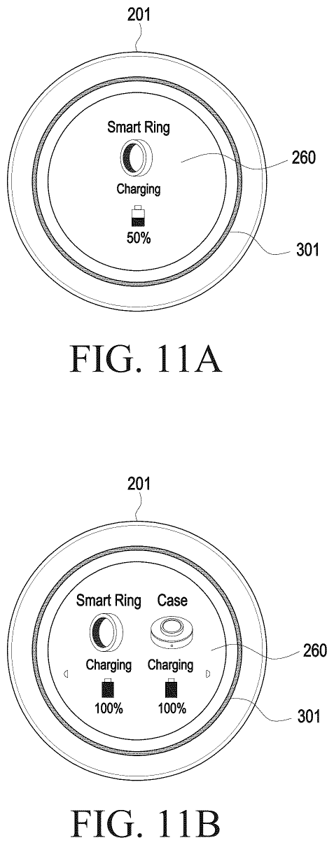

FIG. 11A illustrates a screen for displaying state information of a second battery of a wearable electronic device on a display by an electronic device according to an embodiment.

Referring to FIG. 11A, according to an embodiment, an electronic device 201 (e.g., the electronic device 201 of FIG. 3) may display second state information of a second battery (e.g., the second battery 330 of FIG. 3) of a wearable electronic device 301 (e.g., the wearable electronic device 301 of FIG. 3) on a display 260 (e.g., the display 260 of FIG. 3). For example, the second state information of the second battery 330 may include information (e.g., smart ring, 50%) on a remaining amount of the second battery 330 and information (e.g., smart ring, charging) indicating that the second battery 330 is being charged.

FIG. 11B illustrates a screen for displaying first state information of a first battery and second state information of a second battery of an electronic device on a display by the electronic device according to an embodiment.

Referring to FIG. 11B, an electronic device 201 (e.g., the electronic device 201 of FIG. 3) may display second state information of a second battery 330 (e.g., the second battery 330 of FIG. 3) of a wearable electronic device 301 (e.g., the wearable electronic device 301 of FIG. 3) and first state information of a first battery 230 (e.g., the first battery 230 of FIG. 3) on a display 260 (e.g., the display 260 of FIG. 3). For example, the state information of the second battery 330 may include information (e.g., smart ring, 100%) on a remaining amount of the second battery 330 and information (e.g., smart ring, charging) indicating that the second battery 330 is being charged. For example, the state information of the first battery 230 may include information (e.g., case, 100%) on a remaining amount of the first battery 230 and information (e.g., case, charging) indicating that the first battery 230 is being charged.

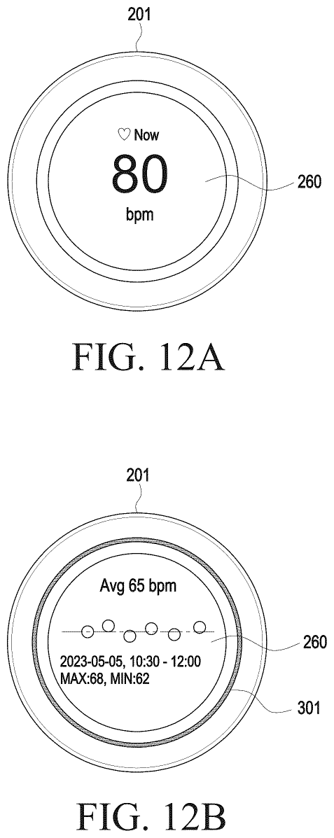

FIG. 12A illustrates a screen for displaying a heart rate of a user wearing a wearable electronic device on a display by an electronic device according to an embodiment.

Referring to FIG. 12A, according to an embodiment, an electronic device 201 (e.g., the electronic device 201 of FIG. 3) may obtain biometric information from a wearable electronic device 301 (e.g., the wearable electronic device 301 of FIG. 3) at each specified time or in real time. According to an embodiment, the biometric information may include a heart rate.

According to an embodiment, the electronic device 201 may display the heart rate obtained from the wearable electronic device 301 on a display 260 (e.g., the display 260 of FIG. 3).

For example, the wearable electronic device 301 may transmit information on the heart rate to the electronic device 201 at each specified time. The electronic device 201 may update the information on the heart rate at each specified time and display the same on the display 260.

For example, the wearable electronic device 301 may transmit the information on the heart rate to the electronic device 201 in real time. The electronic device 201 may update the information on the heart rate and display the same on the display 260 in real time.

FIG. 12B illustrates a screen for displaying a heart rate of a user on a display by an electronic device in a state in which a wearable electronic device is mounted in the electronic device according to an embodiment.

Referring to FIG. 12B, according to an embodiment, an electronic device 201 (e.g., the electronic device 201 of FIG. 3) may identify that a wearable electronic device 301 (e.g., the wearable electronic device 301 of FIG. 3) is mounted in the electronic device 201.

According to an embodiment, the electronic device 201 may obtain, from the wearable electronic device 301, biometric information obtained in a time interval between a time point at which the wearable electronic device 301 is worn by a user and a time point at which the wearable electronic device 301 is mounted in the electronic device 201. For example, the biometric information may include information on at least one of an average heart rate (e.g., 65 bpm), a maximum heart rate (e.g., MAX: 68), or a minimum heart rate (e.g., MIN: 62).

According to an embodiment, the electronic device 201 may display biometric information and information (e.g., 10:30 to 12:00) in a time interval between a time point at which the wearable electronic device is worn by the user and a time point at which the wearable electronic device 301 is mounted in the electronic device 201.

According to an embodiment, the electronic device 201 may display, through the display 260, heart rate values obtained in a specific time period included in a time interval between a time point at which the wearable electronic device is worn by the user and a time point at which the wearable electronic device 301 is mounted in the electronic device 201 and a graph through which the heart rate values are visualized.

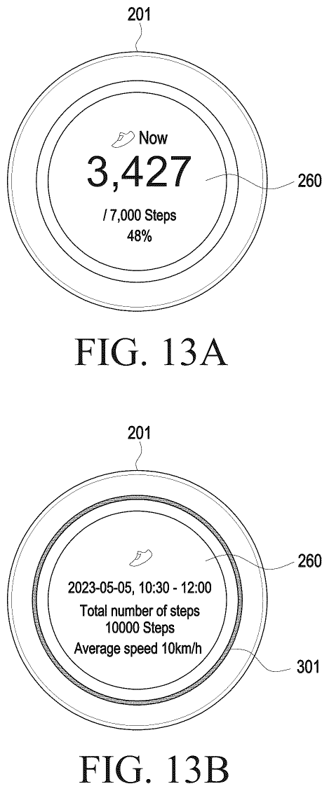

FIG. 13A illustrates a screen for displaying the number of steps of a user wearing a wearable electronic device on a display by an electronic device according to an embodiment.

Referring to FIG. 13A, according to an embodiment, an electronic device 201 (e.g., the electronic device 201 of FIG. 3) may obtain an acceleration value from a wearable electronic device 301 (e.g., the wearable electronic device 301 of FIG. 3) at each specified time or a in real time. According to an embodiment, the electronic device 201 may obtain the number of steps of the user, obtained based on the acceleration value. According to an embodiment, the electronic device 201 may obtain the number of steps of the user from the wearable electronic device 301 at each specified time or in real time.

According to an embodiment, the electronic device 201 may display a pre-configured target number of steps (e.g., 7000) and the number of steps of the user (e.g., 3427) on a display 260 (e.g., the display 260 of FIG. 3). According to an embodiment, the electronic device 201 may display a ratio (e.g., about 48%) of the pre-configured target number of steps to the number of steps of the user.

For example, the electronic device 201 may update information on the pre-configured target number of steps, the number of steps of the user, and the ratio of the preconfigured target number of steps to the number of steps of the user and display the same on the display 260 at each specified time.

For example, the electronic device 201 may update information on the pre-configured target number of steps, the number of steps of the user, and the ratio of the preconfigured target number of steps to the number of steps of the user and display the same on the display 260 in real time.

FIG. 13B illustrates a screen for displaying the number of steps of a user on a display by an electronic device in a state in which a wearable electronic device is mounted in the electronic device according to an embodiment.

Referring to FIG. 13B, according to an embodiment, an electronic device 201 (e.g., the electronic device 201 of FIG. 3) may identify that a wearable electronic device 301 (e.g., the wearable electronic device 301 of FIG. 3) is mounted in the electronic device 201.

According to an embodiment, the electronic device 201 may obtain, from the wearable electronic device 301, information on at least one of an average speed or the number of steps obtained in a time interval between a time point at which the wearable electronic device 301 is worn by the user and a time point at which the wearable electronic device 301 is mounted in the electronic device 201.

According to an embodiment, the electronic device 201 may display biometric information and information (e.g., 10:30 to 12:00) on the time interval between the time point at which the wearable electronic device is worn by the user and the time point at which the wearable electronic device 301 is mounted in the electronic device 201.

According to an embodiment, the electronic device 201 may display, through the display 260, the average speed or the number of steps obtained in a specific time period included in the time interval between the time point at which the wearable electronic device is worn by the user and the time point at which the wearable electronic device 301 is mounted in the electronic device 201.

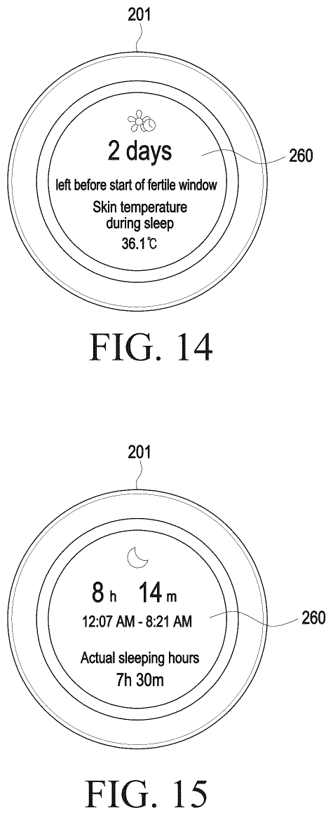

FIG. 14 illustrates a screen for displaying a skin temperature and a fertile window of a user wearing a wearable electronic device on a display by an electronic device according to an embodiment.

Referring to FIG. 14, according to an embodiment, based on biometric information, an electronic device 201 (e.g., the electronic device 201 of FIG. 3) may obtain, from a wearable electronic device 301 (e.g., the wearable electronic device 301 of FIG. 3) information on a fertile window of a user. For example, the wearable electronic device 301 may predict, based on a biometric signal, the fertile window of the user.

According to an embodiment, the wearable electronic device 301 may obtain a skin temperature of the user through a temperature sensor included in the wearable electronic device 301 when the user is sleeping. According to an embodiment, the electronic device 201 may obtain information on the skin temperature of the user from the wearable electronic device 301.

According to an embodiment, the wearable electronic device 301 may display information (e.g., 2 days left before the fertile window) on the fertile window of the user and the information (e.g., about 36.1° C.) on the skin temperature during the sleep on a display 260 (e.g., the display 260 of FIG. 3).

FIG. 15 illustrates a screen for displaying sleeping hours of a user wearing a wearable electronic device on a display by an electronic device according to an embodiment.

Referring to FIG. 15, according to an embodiment, a wearable electronic device 301 (e.g., the wearable electronic device 301 of FIG. 3) may be configured to be in a sleep mode in a state in which the wearable electronic deice 301 is not mounted in an electronic device 201 (e.g., the electronic device 201 of FIG. 3). According to an embodiment, the wearable electronic device 301 may obtain sleeping hours of a user and transmit the obtained sleeping hours to the electronic device 201.

According to an embodiment, the electronic device 201 may display the sleeping hours (e.g., about 7 hours and 30 minutes) of the user and pre-configured target sleeping hours (e.g., 8 hours and 14 minutes) on a display 260 (e.g., the display 260 of FIG. 3). For example, the pre-configured target sleeping hours may be automatically configured by the electronic device 201, or may be configured by the user.

According to an embodiment, the electronic device 201 may output notification information through speaker 212 (e.g., the speaker 212 of FIG. 3) and the display 260 when identification that the sleeping hours of the user correspond to the pre-configured target sleeping hours is made. According to an embodiment, the electronic device 201 may transmit, to the wearable electronic device 301, a control signal for causing the wearable electronic device 301 to output the notification information using a tactile or acoustic means when identification that the sleeping hours of the user correspond to the pre-configured target sleeping hours is made.

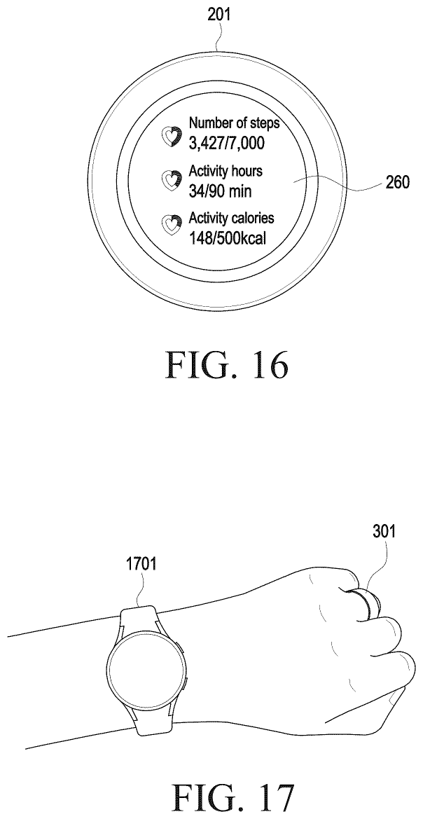

FIG. 16 illustrates a screen for displaying biometric information of a user wearing a wearable electronic device on a display by an electronic device according to an embodiment.

Referring to FIG. 16, according to an embodiment, an electronic device 201 (e.g., the electronic device 201 of FIG. 3) may obtain, from a wearable electronic device 301 (e.g., the wearable electronic device 301 of FIG. 3), information on the number of steps, activity hours of a user, and consumed calories.

According to an embodiment, the electronic device 201 may display, on a display 260 (e.g., the display 260 of FIG. 3) a pre-configured target number of steps (e.g., 7000) compared to the number of steps of the user (e.g., 3427).

According to an embodiment, the electronic device 201 may display preconfigured activity hours (e.g., 90 minutes) compared to activity hours (e.g., about 34 hours) of the user on the display 260.

According to an embodiment, the electronic device 201 may display pre-configured consumed calories (e.g., 500 kcal) compared to consumed calories (e.g., about 148 kcal) on the display 260.

FIG. 17 illustrates a state in which an external electronic device is worn on a wrist part of a user and a wearable electronic device is worn on a finger part of the user according to an embodiment.

Referring to FIG. 17, according to an embodiment, an external electronic device 1701 may be worn on a wrist part of a user. The external electronic device 1701 may be implemented as a wearable electronic device which can be worn on the wrist part of the user. According to an embodiment, a wearable electronic device 301 (e.g., the wearable electronic device 301) may be worn on the wrist port of the user.

According to an embodiment, the wearable electronic device 301 may be communicatively connected to the external electronic device 1701.

According to an embodiment, the external electronic device 1701 may obtain state information of a second battery 330 (e.g., the second battery 330 of FIG. 3) from the wearable electronic device 301. According to an embodiment, the external electronic device 201 may display at least one of state information of a battery of the external electronic device or state information of the second battery 330 on a display according to a user input.

According to an embodiment, the external electronic device 1701 may obtain biometric information of the user from the wearable electronic device 301, and may display the biometric information on the display. For example, the biometric information may include information on a heart rate, ECG, body fat, a skin temperature, a fertile window, sleeping hours, activity hours, or consumed calories.

According to an embodiment, the external electronic device 1701 may display at least one object capable of executing at least one function related to the wearable electronic device 301 on the display. For example, the at least one function may include a function of turning on or off the wearable electronic device 301, a function of initializing the wearable electronic device 301, a function of communicatively connecting the wearable electronic device 301 to a device different from the external electronic device, a function of updating software of the wearable electronic deice 301, or a function of switching the mode of the wearable electronic device 301 to an airplane mode.

According to an embodiment, when an input to at least one object is identified, the external electronic device 1701 may transmit, to the wearable electronic device 301, a control signal for causing at least one function corresponding to the at least one object to be executed. According to an embodiment, when the control signal is received, the wearable electronic device 301 may execute the at least one function.

According to an embodiment, substantially the same operations of the electronic device 201 described in FIGS. 1 to 16, may be also applied to a case of the external electronic device 1701.

According to an embodiment, an electronic device 201 may include a first battery 230, charging circuitry 240, communication circuitry 290, a display 260, a processor 220, and memory storing instructions.

According to an embodiment, the electronic device 201 may transmit, based on identifying that a wearable electronic device 301 is mounted in the electronic device 201, transmit, using the charging circuitry 240, power wirelessly to the wearable electronic device 301.

According to an embodiment, the electronic device 201 may display, on the display 260, at least one of first state information of the first battery or second state information of a second battery included in the wearable electronic device 301 while transmitting the power wirelessly.

According to an embodiment, the electronic device 201 may display, on the display 260, at least one object corresponding to at least one function related to the wearable electronic device 301.

According to an embodiment, the electronic device 201 may transmit, when an input to a first object among the at least one object is identified, through the communication circuitry 290, a control signal to cause the wearable electronic device to execute a first function corresponding to the first object to the wearable electronic device.

According to an embodiment, based on identifying that the wearable electronic device 301 is power on state, the electronic device 201 may display the second state information, or display the first state information and the second state information according to a user input.

According to an embodiment, in the electronic device 201, the at least one function may include a function of turning on or off a power of the wearable electronic device 301, a function of initializing the wearable electronic device 301, or a function of communicatively connecting the wearable electronic device 301 to an external electronic device different from the electronic device 201.

According to an embodiment, the electronic device 201 may display, based on identifying that the wearable electronic device 301 is mounted in the electronic device, on the display, biometric information of a user obtained between a time when the wearable electronic device 301 is identified to be worn by the user and a time when the wearable electronic device is identified to be mounted in the electronic device.

According to an embodiment, the electronic device 201 may store, when the input to the first object capable of executing a function of initializing the wearable electronic device 301 is identified, the biometric information in the memory.

According to an embodiment, the electronic device 201 may transmit a control signal to cause the wearable electronic device to initialize the wearable electronic device 301 to the wearable electronic device 301.

According to an embodiment, the electronic device 201 may obtain, through the communication circuitry 290, the second state information of the second battery 330 from the wearable electronic device 301 in a state in which the wearable electronic device 301 is not mounted in the electronic device 201.

According to an embodiment, the electronic device 201 may display, on the display 260, the second state information in the state in which the wearable electronic device 301 is not mounted in the electronic device.

According to an embodiment, the electronic device 201 may transmit, when identifying that a remaining amount of the second battery 330 is less than a specified value based on the second state information, a control signal to cause the wearable electronic device 301 to output notification information to the wearable electronic device 301.

According to an embodiment, the electronic device 201 may include a microphone 211.

According to an embodiment, the electronic device 201 may transmit, based on obtaining a voice signal for executing a second function among the at least one function through the microphone 211, a control signal to cause the wearable electronic device 301 to execute the second function to the wearable electronic device 301.

According to an embodiment, the electronic device 201 may display, on the display, biometric information of a user obtained from the wearable electronic device 301 at a first time in a state in which the wearable electronic device 301 is not mounted in the electronic device 201.

According to an embodiment, the electronic device 201 may display, when second biometric information of the user obtained at a second time after the first time is obtained from the wearable electronic device in the state in which the wearable electronic device 301 is not mounted in the electronic device 201, on the display, the second biometric information.

According to an embodiment, the electronic device 201 may transmit, when identifying that the user is exercising based on the biometric information, a control signal for causing the wearable electronic device 301 to adjust a measurement period of a sensor 310 included in the wearable electronic device 301 to a second period shorter than a first specified period to the wearable electronic device 301.

According to an embodiment, a method for operating an electronic device 201 may include based on identifying that a wearable electronic device 301 is mounted in the electronic device 201, wirelessly transmitting, using charging circuitry 240 included in the electronic device 201, power to the wearable electronic device 301.

According to an embodiment, the method for operating the electronic device 201 may include displaying, on a display 260 included in the electronic device 201, at least one of first state information of a first battery 230 included in the electronic device 201 or second state information of a second battery 330 included in the wearable electronic device 301 while wirelessly transmitting the power.

According to an embodiment, the method for operating the electronic device 201 may include displaying, on the display 260, at least one object corresponding to at least one function related to the wearable electronic device 301.

According to an embodiment, the method for operating the electronic device 201 may include when an input to a first object among the at least one object is identified, transmitting a control signal to cause the wearable electronic device 301 to execute a first function corresponding to the first object to the wearable electronic device 301 through communication circuitry 290 included in the electronic device 201.

According to an embodiment, the method for operating the electronic device 201 may include based on identifying that the wearable electronic device 301 is in a power on state, displaying the first state information, or displaying the first state information and the second state information according to a user input.

According to an embodiment, in the method for operating the electronic device 201, the at least one function may include a function of turning on or off power of the wearable electronic device 301, a function of initializing the wearable electronic device 301, or a function of communicatively connecting the wearable electronic device 301 to an external electronic device different from the electronic device 201.

According to an embodiment, the method for operating the electronic device 201 may include based on identifying that the wearable electronic device 301 is mounted in the electronic device 201, displaying, on the display 260, biometric information of a user, obtained between a time when the wearable electronic device is identified to be worn by the user and a time when the wearable electronic device is identified to be mounted in the electronic device 201.

According to an embodiment, the method for operating the electronic device 201 may include when the input to the first object capable of executing a function of initializing the wearable electronic device 301 is identified, storing the biometric information in memory included in the electronic device 201.

According to an embodiment, the method for operating the electronic device 201 may include transmitting a control signal to cause the wearable electronic device 301 to initialize the wearable electronic device to the wearable electronic device 301.

According to an embodiment, the method for operating the electronic device 201 may include obtaining the second state information of the second battery 330 from the wearable electronic device 301 through the communication circuitry 290 included in the electronic device 201 in a state in which the wearable electronic device 301 is not mounted in the electronic device.

According to an embodiment, the method for operating the electronic device 201 may include displaying the second state information on the display 260 in the state in which the wearable electronic device 301 is not mounted in the electronic device 201.

According to an embodiment, the method for operating the electronic device 201 may include when identifying that a remaining amount of the second battery 330 is less than a specified value, based on the second state information, transmitting a control signal for causing the wearable electronic device 301 to output notification information to the wearable electronic device 301.

According to an embodiment, the method for operating the electronic device 201 may include based on obtaining a voice signal for executing a second function among the at least one function through a microphone 211 included in the electronic device 201, transmitting a control signal to cause the wearable electronic device 301 to execute the second function to the wearable electronic device 301.

According to an embodiment, the method for operating the electronic device 201 may include displaying, on the display, first biometric information of a user, obtained from the wearable electronic device 301 at a first time in a state in which the wearable electronic device 301 is not mounted in the electronic device 201.

According to an embodiment, the method for operating the electronic device 201 may include when second biometric information of the user is obtained at a second time after the first time from the wearable electronic device in the state in which the wearable electronic device 301 is not mounted in the electronic device 201, displaying, on the display 260 included in the electronic device 201, the second biometric information.

According to an embodiment, a non-transitory computer-readable recording medium may store at least one instruction which, when executed by at least one processor of an electronic device, individually and/or collectively, cause the electronic device to perform at least one operation including, based on identifying that a wearable electronic device is mounted in an electronic device 201, wirelessly transmitting, using charging circuitry 240 included in the electronic device, power to the wearable electronic device.

According to an embodiment, the non-transitory computer-readable recording medium may store at least one instruction which, when executed by at least one processor of an electronic device, individually and/or collectively, cause the electronic device to perform at least one operation including, displaying, on a display 260, at least one of first state information of a first battery 230 included in the electronic device 201 or second state information of a second battery 330 included in the wearable electronic device 301 while wirelessly transmitting the power.

According to an embodiment, the non-transitory computer-readable recording medium may store instruction which, when executed by at least one processor of an electronic device, individually and/or collectively, cause the electronic device to perform at least one operation including, displaying, on the display 260, at least one object capable of executing at least one function related to the wearable electronic device 301.

According to an embodiment, the non-transitory computer-readable recording medium may store at least one instruction which, when executed by at least one processor of an electronic device, individually and/or collectively, cause the electronic device to perform at least one operation including, based on identifying an input to a first object among the at least one object, transmitting a control signal to cause the wearable electronic device 301 to execute a first function corresponding to the first object to the wearable electronic device 301 through communication circuitry 290 included in the electronic device 201.

According to an embodiment, the non-transitory computer-readable recording medium may store at least one instruction which, when executed by at least one processor of an electronic device, individually and/or collectively, cause the electronic device to perform at least one operation including, based on identifying that the wearable electronic device 301 is in a power on state, displaying the first state information, or displaying the first state information and the second state information according to a user input.

According to an embodiment, in the non-transitory computer-readable recording medium, the at least one function may include a function of turning on or off power of the wearable electronic device 301, a function of initializing the wearable electronic device 301, or a function of communicatively connecting the wearable electronic device 301 to an external electronic device different from the electronic device 201.

According to an embodiment, the non-transitory computer-readable recording medium may store at least one instruction which, when executed by at least one processor of an electronic device, individually and/or collectively, cause the electronic device to perform at least one operation including, based on identifying that the wearable electronic device 301 is mounted in the electronic device 201, displaying, on the display 260, biometric information of a user, obtained between a time when the wearable electronic device 301 is identified to be worn by the user and a time when the wearable electronic device is identified to be mounted in the electronic device 201.

According to an embodiment, the non-transitory computer-readable recording medium may store at least one instruction which, when executed by at least one processor of an electronic device, individually and/or collectively, cause the electronic device to perform at least one operation including, based on identifying the input to the first object capable of executing a function of initializing the wearable electronic device 301, storing the biometric information in memory included in the electronic device 201.

According to an embodiment, the non-transitory computer-readable recording medium may store at least one instruction which, when executed by at least one processor of an electronic device, individually and/or collectively, cause the electronic device to perform at least one operation including, transmitting a control signal for causing the wearable electronic device 301 to initialized the wearable electronic device to the wearable electronic device 301.

According to an embodiment, the non-transitory computer-readable recording medium may store at least one instruction which, when executed by at least one processor of an electronic device, individually and/or collectively, cause the electronic device to perform at least one operation including, obtaining the second state information of the second battery 330 from the wearable electronic device 301 through the communication circuitry 290 included in the electronic device 201 in a state in which the wearable electronic device 301 is not mounted in the electronic device 201.

According to an embodiment, the non-transitory computer-readable recording medium may store at least one instruction which, when executed by at least one processor of an electronic device, individually and/or collectively, cause the electronic device to perform at least one operation including, displaying the second state information on the display 260 in the state in which the wearable electronic device 301 is not mounted in the electronic device 201.

According to an embodiment, the non-transitory computer-readable recording medium may store at least one instruction which, when executed by at least one processor of an electronic device, individually and/or collectively, cause the electronic device to perform at least one operation including, based on identifying that a remaining amount of the second battery 330 is less than a specified value, based on the second state information, transmitting a control signal to cause the wearable electronic device 301 to output notification information to the wearable electronic device 301.

According to an embodiment, the non-transitory computer-readable recording medium may store at least one instruction which, when executed by at least one processor of an electronic device, individually and/or collectively, cause the electronic device to perform at least one operation including, based on obtaining a voice signal for executing a second function among the at least one function through a microphone 211 included in the electronic device 201, transmitting a control signal to cause the wearable electronic device 301 to execute the second function to the wearable electronic device 301.

According to an embodiment, the non-transitory computer-readable recording medium may store at least one instruction which, when executed by at least one processor of an electronic device, individually and/or collectively, cause the electronic device to perform at least one operation including, displaying, on the display, first biometric information of a user, obtained from the wearable electronic device 301 at a first time in a state in which the wearable electronic device 301 is not mounted in the electronic device 201.

According to an embodiment, the non-transitory computer-readable recording medium may store at least one instruction which, when executed by at least one processor of an electronic device, individually and/or collectively, cause the electronic device to perform at least one operation including, based on obtaining second biometric information of the user at a second time after the first time from the wearable electronic device in the state in which the wearable electronic device 301 is not mounted in the electronic device 201, displaying, on the display 260, the second biometric information.

The electronic device according to various embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101, 201, 301, 1701). For example, a processor (e.g., the processor 120, 220, 320) of the machine (e.g., the electronic device 101, 201, 301, 1701) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added.

Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. An electronic device, comprising:
a first battery;
charging circuitry;
communication circuitry;
a display;
at least one processor; and
memory storing instructions, that, when executed by the processor, individually and/or collectively, cause the electronic device to:
based on identifying that a wearable electronic device is mounted in the electronic device, transmit, using the charging circuitry, power wirelessly to the wearable electronic device,
display, on the display, at least one of first state information of the first battery or second state information of a second battery included in the wearable electronic device while transmitting the power wirelessly,
display, on the display, at least one object corresponding to at least one function related to the wearable electronic device, and
based on identifying an input to a first object among the at least one object, transmit, through the communication circuitry, a control signal to cause the wearable electronic device to execute a first function corresponding to the first object to the wearable electronic device.

2. The electronic device of claim 1, wherein the instructions, when executed by the at least one processor, individually and/or collectively, cause the electronic device to:
based on identifying that the wearable electronic device is in a power on state, display, on the display, the first state information, or display, on the display, the first state information and the second state information according to an input.

3. The electronic device of claim 1, wherein the at least one function includes a function of turning on or off power of the wearable electronic device, a function of initializing the wearable electronic device, and/or a function of communicatively connecting the wearable electronic device to an external electronic device different from the electronic device.

4. The electronic device of claim 1, wherein the instructions, when executed by the at least one processor, individually and/or collectively, cause the electronic device to:
based on identifying that the wearable electronic device is mounted in the electronic device, display, on the display, biometric information of a user obtained between a time when the wearable electronic device is identified to be worn by the user and a time when the wearable electronic device is identified to be mounted in the electronic device.

5. The electronic device of claim 4, wherein the instructions, when executed by the at least one processor, individually and/or collectively, cause the electronic device to:
based on identifying the input to the first object capable of executing a function of initializing the wearable electronic device, store the biometric information in the memory, and
transmit a control signal to cause the wearable electronic device to initialize the wearable electronic device to the wearable electronic device.

6. The electronic device of claim 1, wherein the instructions, when executed by the at least one processor, individually and/or collectively, cause the electronic device to:
obtain, through the communication circuitry, the second state information of the second battery from the wearable electronic device in a state in which the wearable electronic device is not mounted in the electronic device, and
display, on the display, the second state information in the state in which the wearable electronic device is not mounted in the electronic device.

7. The electronic device of claim 6, wherein the instructions, when executed by the at least one processor, individually and/or collectively, cause the electronic device to:
based on identifying that a remaining amount of the second battery is less than a specified value based on the second state information, transmit a control signal to cause the wearable electronic device to output notification information to the wearable electronic device.

8. The electronic device of claim 1, further comprising:
a microphone, and
wherein the instructions, when executed by the at least one processor, individually and/or collectively, cause the electronic device to: based on obtaining a voice signal for executing a second function among the at least one function through the microphone, transmit a control signal to cause the wearable electronic to execute the second function to the wearable electronic device.

9. The electronic device of claim 1, wherein the instructions, when executed by the at least one processor, individually and/or collectively, cause the electronic device to:
display, on the display, first biometric information of a user obtained from the wearable electronic device at a first time in a state in which the wearable electronic device is not mounted in the electronic device, and
based on obtaining second biometric information of the user obtained at a second time after the first time from the wearable electronic device in the state in which the wearable electronic device is not mounted in the electronic device, display, on the display, the second biometric information.

10. The electronic device of claim 9, wherein the instructions, when executed by the at least one processor, individually and/or collectively, cause the electronic device to:
based on identifying that the user is exercising based on the biometric information, transmit a control signal to cause the wearable electronic device to adjust a measurement period of a sensor included in the wearable electronic device to a second period shorter than a first specified period to the wearable electronic device.

11. A method of operating an electronic device, the method comprising:
based on identifying that a wearable electronic device is mounted in the electronic device, wirelessly transmitting, using charging circuitry included in the electronic device, power to the wearable electronic device;
displaying, on a display included in the electronic device, at least one of first state information of a first battery included in the electronic device or second state information of a second battery included in the wearable electronic device while wirelessly transmitting the power;
displaying, on the display, at least one object corresponding to at least one function related to the wearable electronic device; and
based on identifying an input to a first object among the at least one object, transmitting, through communication circuitry included in the electronic device, a control signal to cause the wearable electronic device to execute a first function corresponding to the first object to the wearable electronic device.

12. The method of claim 11, further comprising based on identifying that the wearable electronic device is in a power on state, displaying the first state information, or displaying the first state information and the second state information according to an input.

13. The method of claim 11, wherein the at least one function includes a function of turning on or off power of the wearable electronic device, a function of initializing the wearable electronic device, and/or a function of communicatively connecting the wearable electronic device to an external electronic device different from the electronic device.

14. The method of claim 11, further comprising based on identifying that the wearable electronic device is mounted in the electronic device, displaying, on the display, biometric information of a user, obtained between a time when the wearable electronic device is identified to be worn by the user and a time when the wearable electronic device is identified to be mounted in the electronic device.

15. The method of claim 14, further comprising:
based on identifying the input to the first object capable of executing a function of initializing the wearable electronic device, storing the biometric information in memory included in the electronic device; and
transmitting a control signal to cause the wearable electronic device to be initialized to the wearable electronic device.

16. The method of claim 11, further comprising:
obtaining, through the communication circuitry included in the electronic device, the second state information of the second battery from the wearable electronic device in a state in which the wearable electronic device is not mounted in the electronic device; and
displaying the second state information on the display in the state in which the wearable electronic device is not mounted in the electronic device.

17. The method of claim 16, further comprising based on identifying that a remaining amount of the second battery is less than a specified value, based on the second state information, transmitting a control signal to cause the wearable electronic device to output notification information to the wearable electronic device.

18. The method of claim 11, wherein the electronic device further comprises a microphone, and
the method for operating the electronic device further comprises: based on obtaining a voice signal for executing a second function among the at least one function through the microphone, transmitting a control signal to cause the wearable electronic device to execute the second function to the wearable electronic device.

19. The method of claim 11, further comprising:
displaying, on the display, first biometric information of a user, obtained from the wearable electronic device at a first time in a state in which the wearable electronic device is not mounted in the electronic device; and
based on second biometric information of the user being obtained at a second time after the first time from the wearable electronic device in the state in which the wearable electronic device is not mounted in the electronic device, displaying, on the display, the second biometric information.

20. A non-transitory computer-readable recording medium configured to store instructions which, when executed by at least one processor, individually and/or collectively, of an electronic device, cause the electronic to perform at least one operation comprising:
based on identifying that a wearable electronic device is mounted in the electronic device, wirelessly transmitting, using charging circuitry included in the electronic device, power to the wearable electronic device;
displaying, on a display included in the electronic device, at least one of first state information of a first battery included in the electronic device or second state information of a second battery included in the wearable electronic device while wirelessly transmitting the power,
displaying, on the display, at least one object capable of executing at least one function related to the wearable electronic device; and
based on identifying an input to a first object among the at least one object, transmitting, through communication circuitry included in the electronic device, a control signal to cause the wearable electronic device to execute a first function corresponding to the first object to the wearable electronic device.
